(19) United States
(12) Patent Application Publication
(10) Pub. No.: US 2008/0286379 A1
(43) Pub. Date: Nov. 20, 2008

(54) Method and Means for Obtaining Platelet-Rich Plasma

(76) Inventors: Peter Wehling, Dusseldorf (DE); Julio Reinecke, Koln (DE)

Correspondence Address: HARNESS, DICKEY & PIERCE, P.L.C., P.O. BOX 828, BLOOMFIELD HILLS, MI, 48303, US

(21) Appl. No.: 11/658,210

(22) PCT Filed: Jul. 29, 2005
(86) PCT No.: PCT/EP05/08252

§ 371 Date: Jan. 23, 2007

(30) Foreign Application Priority Data
Jul. 29, 2004 (DE) 10 2004 036 840.6

(51) Int. Cl. A61K 35/16; B01D 21/26; B03D 3/02; B01D 29/085

(52) U.S. Cl. 424/530; 210/787; 210/739; 210/696; 210/255

(57) ABSTRACT

Method and means for obtaining thrombocyte-rich plasma (platelet-rich plasma, PRP) from whole blood which specifically has a high content of specifically activated thrombocytes and which is particularly easy to coagulate.

The present invention relates to method and means for obtaining thrombocyte-rich plasma (PRP) which has a high thrombocyte content and which is particularly easily gelable from whole blood.

One of the uses for thrombocyte-rich plasma (PRP) in medicine is in the treatment of wounds to support bone healing and hemostasis, in particular in plastic surgery, etc. The preparation and the use of autologous, thrombocyte-rich plasma from whole blood is pre-eminent.

Thrombocytes in thrombocyte-rich plasma are distinguished in particular by their high protein content, in particular growth factors and cytokines which can be released from the thrombocytes with appropriate treatment. In the case of the growth factors and cytokines, it is primarily PDGF (platelet derived growth factor), TGF (transforming growth factor), VEGF (vascular endothelial growth factor) and EGF (epithelial growth factor). These substances possess a plurality of positive effects on human health, in particular the prophylaxis and therapy of illnesses in the animal and human body. The interest of medicine in methods for obtaining concentrations of thrombocytes or thrombocyte-rich plasma is consequently great.

Slater et al. (J. Orthopaedic Res. 13:655-66) have shown in preclinical tests in human fetal osteoblast-like cells in cell culture that the addition of a human thrombocyte-rich plasma to a cell culture medium increased the thymidine absorption of the cells as an indicator for the synthesis of bone matrix by approximately four-fold. Over a period of 30 days, the thickness of the multi-cell layer formed by about 36-fold compared with comparable untreated cultures.

The first clinical users were Marx et al. (Oral Surg. Oral Med. Oral Pathol. Oral Radiol. Endod (1998) 85:638-646 who showed that the addition of thrombocyte concentrate to autologous bone grafts which were used primarily for the reconstruction of the jawbone resulted in a clearly quantifiable acceleration of bone formation and increased bone thickness in this area compared with untreated bone grafts. Undesirable side effects were not observed. Subsequently Lowery et al. (Bone (August 1999) Suppl. 25(2):47S-50S) conducted successful tests on the effect of autologous thrombocyte concentrate in the fusion of vertebral bodies. They found that bone maturation is clearly accelerated in the initial stage. No side effects were observed here either.

Essentially, the medical use of autologous thrombocyte-rich plasma, in addition to its property of constituting a rich source of different growth factors and cytokine, consists in the promotion of accelerated healing in all tissues, in the promotion of greater collagen concentration in healing wounds which leads particularly to greater scar strength, in the promotion of accelerated new vesicular formation, in the promotion of reduced bone maturation time, in the promotion of increased local bone thickness, in the promotion of partially reduced postoperative pain and the promotion of reduced wound infection rates. The danger of transmitting illness from foreign organisms in particular is eliminated when obtaining the thrombocyte-rich plasma from autologous whole blood.

A known technique for obtaining thrombocyte-rich plasma from whole blood is described in WO 00/61265 and WO 01/83068 from Harvest Technologies Co., wherein a device with two communicating chambers is used, and for the preparation of the thrombocyte-rich plasma the first chamber is filled with drawn whole blood, an erythrocyte-rich pellet and plasma supernatant containing the thrombocytes is separated automatically by centrifugation through the disposition of a float within the chamber system during the centrifugation. A fluctuating hematocrit cannot be compensated for. In a second centrifugation step, plasma and a thrombocyte-rich fraction are separated from each other and then the plasma supernatant is partially removed and the thrombocyte pellet is resuspended in a remaining amount of plasma to obtain a thrombocyte-rich plasma.

A further known method for obtaining thrombocyte-rich plasma from whole blood is described in WO 00/54825, or U.S. Pat. No. 6,325,750, from Implant Innovations Inc., wherein a system having two flexible bags communicating through a bridge is placed in a bucket. The whole blood applied in the one bag is centrifuged and subsequently, by injecting a fixed quantity of air into the cavity between the flexible chamber wall and the solid wall of the bucket, the thrombocyte-containing plasma supernatant is separated from the erythrocyte-rich pellet and transferred to the second flexible bag. Here too, in a second centrifugation step, plasma and a thrombocyte-rich fraction are separated, the plasma supernatant is partially removed and the thrombocyte pellet is resuspended in a remaining percentage of plasma to obtain a thrombocyte-rich plasma.

The known technologies and methods for obtaining autologous, thrombocyte-rich plasma cannot provide a continuous high yield and quality. The fluctuations in yield and quality can be attributed to the variability of the hematocrit of the whole blood drawn. The hematocrit, that is the proportion by volume of the cellular components of the total volume of the whole blood, can fluctuate considerably between individuals, for example from 35 to 50%. The existing systems for producing fresh thrombocyte concentrates are not capable of compensating for this variability in the hematocrit. Consequently, the quality of the end product fluctuates considerably with respect to the yield and the quality of the thrombocyte-rich plasma obtained.

A further cause of the fluctuations in the yield and quality is the sometimes considerable time interval between drawing the blood and preparation of the thrombocyte-rich plasma. In normal production in blood banks, the time span between production and use is too great to permit an acceptable yield of active growth factors. At least 500 ml of whole blood are normally required for preparation. This means that the patient must donate blood at least 5 days in advance for an autologous preparation to keep down the physical stress. The storage then required leads inevitably to a diminution of quality.

It is therefore desirable to be able to produce thrombocyte-rich plasma autologously and as far as possible on site and fresh, as well as under always sterile conditions with an identical continuous yield. It is furthermore desirable to obtain a thrombocyte-rich plasma of high quality which forms a large quantity of useful proteins such as growth factors and cytokines and has a high proportion of thrombocytes.

Additionally, thrombocyte-rich plasma is being increasingly as a coagulated thrombocyte-rich gel used for a number of applications. The mechanical strength of the gel is critical to its effectiveness and efficacy in this use. It turns out however that thrombocyte-rich plasma obtained using known methods is also of low quality with respect to its clotting ability. It is therefore also desirable to obtain thrombocyte-rich plasma which has high clotting ability and can be coagulated into a thrombocyte-rich gel which can be better used.

The technical problem underlying the present invention consists essentially in preparing a method and means for obtaining thrombocyte-rich plasma from whole blood which overcome the disadvantages in the prior art whereby the thrombocyte-rich plasma obtained is rich in thrombocytes, in particular activated thrombocytes, and/or has improved clotting ability.

The present invention is solved in accordance with the invention by preparing a method for obtaining plasma rich in thrombocytes from whole blood wherein in a first step a) the whole blood is separated into at least one fraction containing erythrocytes and a plasma fraction which contains the thrombocytes and is essentially free of erythrocytes, in a second step b) the plasma containing thrombocytes is separated from the fraction containing erythrocytes, in a further step c) preferably by means of centrifugation, the plasma containing thrombocytes is separated into a fraction containing thrombocytes which, preferably in addition to the thrombocytes, also has nuclear cells, that is mononuclear cells (buffy coat) and/or specifically is present as a pellet, wherein preferably still further thrombocytes are to some extent present suspended in plasma directly above the pellet formed, and into a supernatant of thrombocyte-poor plasma, in a further step d) the supernatant obtained from thrombocyte-poor plasma is removed, preferably by means of a syringe, in a further step e) the thrombocyte-poor plasma removed is thoroughly mixed, in a further step f) mixed thrombocyte-poor plasma is returned to the thrombocyte fraction, that is reapplied, in a further step g) the thrombocyte fraction is suspended in the reapplied thrombocyte-poor plasma, in particular by thorough mixing , preferably by mechanical mixing and in a further step h) the resuspended thrombocyte fraction in the reapplied thrombocyte-poor plasma is obtained as a thrombocyte-rich plasma, in particular as plasma rich in activated thrombocytes.

The inventors found it surprising that specifically through the reapplication of previously removed plasma supernatant, thoroughly mixed after its removal, clearly increased thrombocyte activation results. The inventors found it additionally surprising that specifically through the reapplication of previously removed plasma supernatant, thoroughly mixed after its removal, a thrombocyte-rich plasma is obtained which has definitely improved clotting ability. Without being restricted to the theory, this effect can probably be attributed to the thrombocytes being resuspended through this procedural step in accordance with the invention in a plasma which contains all plasma components, of high molecular weight and low molecular weight, in the physiologically correct composition. The clotting factors important for the subsequent coagulation are thereby also contained in the thrombocyte-rich plasma obtained in accordance with the invention, whereby this can be more easily coagulated and an improved thrombocyte-rich plasma can be obtained.

The known methods do not achieve this since only one part of the plasma is extracted here from the supernatant to obtain a thrombocyte-rich plasma while a remnant of plasma, containing in particular high-molecular weight portions, is mixed with the thrombocyte fraction.

Provision is made in the invention that specifically autologous and specifically venous whole blood is drawn from a patient and immediately afterwards, from this blood, at least an erythrocyte-rich fraction is separated from the plasma containing the thrombocytes and preferably the buffy coat, that is from at least a fraction containing predominantly thrombocytes and mononuclear cells. To do this, the whole blood introduced into a chamber, specifically flexible chamber, preferably a flexible bag, is separated by means of centrifugation, preferably at between 1500 to 3500 rpm, preferably from 2000 to 2800 rpm, over a preferred period of 1.5 to 4 minutes and in the chamber an erythrocyte-rich, specifically an erythrocyte-containing fraction is obtained as a pellet on the bottom of the chamber and at least one buffy coat fraction is obtained as a supernatant containing the thrombocytes and preferably mononuclear cells. At least three fractions are obtained: an erythrocyte-containing fraction, which appears red, a thrombocyte-containing buffy coat fraction which appears as a thin, white, viscous layer, and a supernatant of plasma which has a yellow to orange appearance

In a particularly preferred embodiment, the whole blood fractionated by centrifugation in the chamber, in particular the flexible bag, has mechanical pressure applied by rolling up and/or expressing the chamber such that the supernatant of plasma together with the thrombocyte-containing buffy coat fraction, which are found in the upper section of the chamber, is pressed out of the first chamber and in particular by way of a connecting bridge which is attached to a second chamber, specifically a flexible chamber, specifically a flexible bag, is taken into said chamber, wherein the erythrocyte-containing pellet remains in the first chamber. In accordance with the invention, the fraction containing erythrocyte is separated as a function of the hematocrit of the whole blood that was drawn. Preferably the adjustment is carried out by pressing the supernatant out of the first chamber, specifically by rolling up and/or expression, is continued until the plasma supernatant is largely transferred and the erythrocyte-containing fraction, that is the erythrocyte-containing pellet, is localized at the upper end of the chamber. Through the particularly preferred embodiment of the first chamber in accordance with the invention and/or the transfer line made of an optically transparent material, the incipient transfer of an erythrocyte-containing fraction, and thus the end of the transfer process, can be detected by the appearance of erythrocytes at the upper end of the first chamber and/or in the transfer tube. The invention utilizes the properties of erythrocytes of absorbing light energy, in particular in the visible range of light, in particular through the appearance of a red-to-blue coloration. The presence of erythrocytes at the upper end of the first chamber and/or in the transfer line is determined in accordance with the invention preferably through suitable detectors or counters in a way known per se and/or through a simple visual check and then the transfer process is stopped. In this way, what is advantageously achieved in accordance with the invention is that in each case and independently of the individual hematocrit present, the greatest possible quantity of thrombocytes is obtained.

In conjunction with the present invention, the formulation “controlled transfer” is understood to mean the process of transferring the supernatant of thrombocyte-rich plasma from centrifuged, fractionated whole blood, which as a function of the individually existing hematocrit of the whole blood being used, the process of transferring the plasma supernatant is stopped at the moment of the detectable incipient transfer of an erythrocyte-containing fraction. The incipient transfer of an erythrocyte-containing fraction is preferably characterized by a detectable amount of erythrocyte at the upper end of the first chamber and/or in the transfer line. Preference is given to transferring all thrombocytes into the second chamber as a particularly pure thrombocyte fraction, meaning free from erythrocytes.

The invention further foresees that in additional process steps the transferred and thrombocyte-containing plasma separated from the erythrocyte-containing fraction, preferably by centrifugation at between 2900 and 5000 rpm, preferably at 3200 rpm, over a period of 10 to 20 minutes, preferably of 15 minutes, is fractionated into a thrombocyte fraction, which is present specifically as a pellet preferably together with mononuclear cells, and into a thrombocyte-poor plasma supernatant. In accordance with the invention, thrombocyte-poor plasma is removed specifically through an opening in the upper section of the second chamber, specifically of the flexible bag in which the separation of the thrombocyte-containing plasma took place, so that the thrombocyte fraction, which exists preferably as a pellet, remains in the second chamber. In one variant, the thrombocyte-poor plasma is removed completely and/or essentially. In an alternative variant, up to 90%, 80%, 70%, 60%, 50%, 20%, 10% of the thrombocyte-poor plasma is removed. In a particularly preferred variant, the thrombocyte-poor plasma is removed in a quantity which is chosen as a function of the individual hematocrit of the whole blood used.

After the separation of thrombocytes and plasma, the plasma is stratified, that means small components are found specifically in the upper part of the plasma and larger components are found in the lower part of the plasma, if, as in known methods, only the upper part of the plasma is now removed after centrifugation, the plasma remaining with the thrombocyte fraction is unnaturally enriched with high-molecular weight components. In accordance with the invention, the thrombocyte-poor plasma removed is mechanically mixed, preferably in the means used for the removal, specifically a syringe. What this advantageously achieves in accordance with the invention is that the plasma components separated by the preceding centrifugation are mixed again so that a largely physiological composition of the plasma results, that means a plasma is obtained which has a homogenous composition of naturally occurring plasma. In a further step in accordance with the invention, the thrombocyte-poor plasma, removed and mixed, is returned to the thrombocyte fraction, specifically to the second chamber containing the thrombocyte fraction, that is to say reapplied, and then the thrombocyte fraction is mixed with the returned plasma and thereby resuspended. In accordance with the invention, the resuspension preferably takes place through mechanical mixing of the thrombocyte fraction from the reapplied plasma. In accordance with the invention it is foreseen to perform the resuspension in the second chamber which is preferably designed as a flexible bag, wherein the content of the second chamber is blended preferably by applying manual, mechanical pressure to the flexible chamber walls, specifically by massaging. In accordance with the invention, this advantageously reduces the mechanical stress on the mechanically sensitive thrombocytes to a minimum, which promotes obtaining plasma rich specifically in activated thrombocytes. The plasma which is rich specifically in activated thrombocytes is then obtained by transferring, or absorbing, the thrombocyte fraction resuspended in the reapplied thrombocyte-poor plasma.

In accordance with the invention, it is preferably foreseen to provide the thrombocyte-rich plasma obtained in a further step i) with at least one coagulant, meaning a clotting agent, so that the plasma coagulates and a gel is obtained rich specifically in activated thrombocytes and/or growth factors. The formation of the gel is strongly promoted by the fact that the suitable concentration and the suitable proportion of all clotting factors which are naturally present in plasma, are present in the thrombocyte-rich plasma obtained in accordance with the invention.

Both the thrombocyte-rich plasma obtained in accordance with the invention and the thrombocyte-rich gel preferably obtained in accordance with the invention have, compared with the thrombocyte-rich plasma obtained by known methods, a clearly strengthened formation, or concentration of proteins such as growth factors and/or cytokines in the thrombocytes contained. In particular, depending on the quality of the thrombocytes obtained and/or the coagulation, there is a disintegration of thrombocytes whereby the growth factors, or cytokines, are released as part of gel formation. It has been shown that the following growth factors, or cytokines, are formed more strongly in one embodiment of the present invention in the thrombocyte-rich plasma obtained in accordance with the invention and in particular in the thrombocyte-rich gel obtained therefrom: The growth factors PDGF, TGF-β, HGF, FGF-II and IGF-I and the anti-inflammatory IL1ra. TNFα and IL-1β are inflammation markers which on the other hand are scarcely elevated.

Subjects of the present invention are therefore the plasma produced by means of the method in accordance with the invention which is rich specifically in activated thrombocytes and the gel formed therefrom, particularly through coagulation with a coagulate. Because of their advantageous properties, the thrombocyte-rich plasma obtained in accordance with the invention, or the gel respectively, serve prophylaxis and/or therapy, or the healing of a plurality of illnesses.

Since the thrombocyte-rich plasma in accordance with the invention, or the gel respectively, has an especially advantageous high concentration of leucocytes, it is used in preference to reduce the risk of infection during treatment. Further, the thrombocyte-rich plasma in accordance with the invention, or the gel, has to its special advantage a high concentration of dendritic cells.

The inventors found it further surprising that the thrombocyte-rich plasma, or the gel, obtained in accordance with the invention provides an especially advantageous “adhesive” to fill and/or repair bone defects if it is blended, for example, with autologous bone grafts and/or bone substitute, for example hydroxylapatite. A further subject of the present invention is thus also the use of the thrombocyte-rich plasma, or the gel, obtained in accordance with the invention to fill or repair bone defects in conjunction with bone grafts and/or bone substitutes.

It was further surprising that the thrombocyte-rich plasma, or the gel, obtained in accordance with the invention speeds up the formation of intercellular matrix, which for example, results especially advantageously in earlier wound closure. A further subject of the present invention is therefore also the use of the thrombocyte-rich plasma, or the gel, in accordance with the invention to speed up wound closure.

The clinical areas of use of the method in accordance with the invention and of the thrombocyte-rich plasma, or the gel, obtainable in accordance with the invention are manifold. They include oral, maxillary and facial surgery, orthopedics, plastic and reconstructive surgery and dermatology. A subject of the present invention is also the use of the thrombocyte-rich plasma, or the gel, in accordance with the invention to speed up and/or support the healing of diabetic ulcerations, especially on the lower extremities.

The subject of the present invention is therefore also the use of the thrombocyte-rich plasma and/or gel in accordance with the invention to speed up the regeneration of bones, cartilage defects, endothelium, epithelium and/or epidermis; to stimulate vascularization; to strengthen collagen synthesis; to accelerate the healing of soft tissue; to reduce scar formation; to alleviate hemostasis; to mitigate and/or reverse the negative effects of corticoids on wound healing; when filling cartilage defects in autologous cartilage transplant (ACT) where a matrix with cartilage cells is bonded to the defect, or respectively the use of the stated plasma or gel to produce appropriate pharmaceutical preparations.

A further subject of the present invention is a device, specifically a bag system, which can preferably be used to carry out the method in accordance with the invention. The device comprises at least one primary chamber (10) and at least one secondary chamber (30) which form a communicating chamber system. Primary chamber (10) and secondary chamber (30) are connected through at least one, specifically closable (20) transfer line. In conjunction with the present invention, a “primary chamber” is understood to be a chamber, that is to say container, into which the fluid or suspension to be separated into their individual components is introduced or is present and undergoes an initial fractionation. A “secondary chamber” is understood to be a chamber, that is a container, into which the fluid or suspension separated completely or partially into its individual components in the primary chamber is introduced completely or partially, that is to say individual fractions thereof, and undergoes a secondary fractionation in the secondary chamber. In accordance with the invention, each of these chambers is provided with at least one, specifically closable discharge and/or delivery (11, 31), specifically for the supply, that is to say introduction or reapplication of blood components, and/or removal, that is to say extraction, of blood components. In a particularly preferred embodiment, primary chamber (10), secondary chamber (30) and transfer line (20) are attached to a carrier plate (60). It is particularly preferable that the transfer line (20) can be closed by at least one interrupt (21) which can be configured as a valve, spigot and/or plug. In a particularly preferred variant, the transfer line (20) is designed as a flexible hose and can be closed specifically by at least one interrupt (21) as a clamp, specifically hose clamp or as a slide clamping the hose which is preferably located on the carrier plate (60). Preferably the transfer line (20) is optically permeable, that is to say transparent, preferably optically clear in order to permit optical checking of the transfer of erythrocytes using technical means and/or visual inspection. In a preferred variant, the transfer line (20) is equipped with an optical detector or counter to detect the presence of erythrocytes in the transfer line.

Both primary chamber (10) and secondary chamber (30) are preferably designed as flexible bags in accordance with the invention. These bags are preferably configured in a “pear shape” which is constructed from an essentially semi-circular lower section (14, 34) and an essentially funnel-shaped upper section (15, 35). narrowing towards the top. In an especially preferred variant, these flexible bags are designed as normally initially flat bags by welding or bonding flexible sheets in which the joined sheets preferably lie against each other and the bags normally assume characteristic bag shape when the lumen between the joined sheets is filled as intended.

The secondary chamber (30) is further characterized in accordance with the invention in that at least one riser tube (40) extending into the lumen of the secondary chamber is formed at the at least one discharge and/or delivery of the secondary chamber having at least one lower opening (42) which is configured preferably in the middle of the secondary chamber, preferably on the border between a semi-circular lower section (34) and a funnel-shaped upper section (35) of the secondary chamber and having at least one upper opening (41) which is configured in the upper section of the secondary chamber, preferably at the upper peak of the funnel-shaped upper part of the secondary chamber, in particular at the collar of the discharge and/or delivery (31) on the inside of the wall of the secondary chamber.

The embodiment in accordance with the invention of the primary and secondary chamber in a pear-shaped form, that is with an upwardly tapering funnel-shaped upper section and a semicircular-shaped lower section improved monitoring of the separation of erythrocytes from the thrombocyte-containing buffy coat following fractionation of the whole blood in the primary chamber is advantageously achieved in accordance with the invention. The result of the pear shape in accordance with the invention is that the boundary between erythrocytes, buffy coat and supernatant plasma can be reproduced sharply and clearly. After the first centrifugation, a broad boundary zone (separation zone) exists between erythrocytes and the thrombocyte-containing buffy coat. As a result of the taper formed in accordance with the invention, this broad boundary is narrowed just shortly before the transfer line. The improved monitoring which this allows permits better separation and higher yield. With the devices from the prior art, which do not have the shape in accordance with the invention, the boundary between erythrocytes and thrombocyte-rich buffy coat is not clearly delineated; as a result, it can easily happen that erythrocytes are transferred although a not insubstantial amount of plasma and buffy coat is still present in the primary chamber.

Centrifuging is preferably performed for a second time in accordance with the invention after the transfer of the plasma supernatant from the primary chamber (10) by way of the transfer line (20) into the secondary chamber (30) as preferred in accordance with the invention in order to obtain a thrombocyte fraction and a thrombocyte-poor plasma supernatant. In so doing, the pear shape of the secondary chamber (30) as preferred under the invention allows a particularly favorable, because consistent, distribution of the effects of centrifugal forces on the thrombocytes by volumetric content, whereby the rpm and centrifugation time required for effective fractionation of the blood components can be reduced to a minimum, which results in reduced mechanical stress on the thrombocytes.

In a preferred embodiment, the device in accordance with the invention for centrifugation is placed in a centrifuge bucket which is shaped such that the primary chamber and/or secondary chamber, preferably designed as a flexible bag, is expanded during centrifugation such that the chamber walls come to rest partially and/or completely against the interior wall of the centrifuge bucket. The use of a sterile bucket is preferred. It is particularly advantageous that the expansion load on the chamber walls and the cells contained is reduced during centrifugation. The preferred use of a centrifuge bucket also allows the use of mechanically lighter, thinner and less solid material for the flexible bag preferred in accordance with the invention. The advantage of the lighter and thinner material is also that the blending, that is resuspension, of the thrombocytes with the added plasma is easier.

In a preferred embodiment, primary chamber and secondary chamber of the device in accordance with the invention are made from a material which, because of its surface property and its chemical composition, is particularly advantageous for the thrombocyte-rich plasma obtained by means of the device and its coagulation capability. Maximum activation of the thrombocytes without triggering premature coagulation is of primary importance.

The riser tube (40) assigned in accordance with the invention to the secondary chamber (30) and its at least one discharge and delivery (31) with an upper opening (41) and a lower opening (42) allows the particular advantage of practically complete removal or complete emptying of the secondary chamber since a great part of the supernatant contained in the secondary chamber after centrifugation is initially removed through the lower opening of the riser tube projecting into the lumen of the secondary chamber, and then the final remnant, after rotating the secondary chamber so that the upward tapering peak of the secondary chamber points down, can be removed through the upper opening (41) at the peak of the tapering part of the secondary chamber.

What can be achieved through the combination in accordance with the invention of the aforementioned features of the device in accordance with the invention is that in a short time, in a sterile environment and specifically immediately after the blood is drawn, a great quantity of high-quality thrombocyte-rich plasma can be obtained effectively and with a high yield.

Naturally the device in accordance with the invention also allows other blood components to be obtained, such as serum, erythrocyte concentrate, buffy coat or mononuclear cell concentrate, and thrombocyte-rich plasma. Beyond that, the device is also suitable for the separation of other intercellular or bodily fluids containing specifically cellular components. The device in accordance with the invention can also be used to fractionate all types of cell suspensions, for example, cultivated mammalian cells, into their components and to obtain the fractions, for example cell components, high molecular-weight proteins, etc. separately. The device in accordance with the invention preferably allows optical inspection when separating cellular from non-cellular, or additional cellular components. Provision is also made to mark different cell components of a cell suspension or of a fluid containing cellular components with suitable dyes.

The invention also relates to a specifically sterile packaged kit comprising the device in accordance with the invention. The kit preferably contains at least one consumable material, preferably all consumable materials which are needed for the production of thrombocyte-rich plasma from whole blood by means of the device in accordance with the invention. The system is simple to use and can be used directly at the site of the intervention. In addition to the aforementioned device in accordance with the invention, commercially available disposable articles such as syringes, cannulas, clamps, etc. are preferably used. A subject of the present invention is therefore also a device, specifically a kit, to obtain plasma specifically rich in activated thrombocytes from whole blood containing at least one means to separate the whole blood into an erythrocyte-containing fraction and thrombocyte-containing and essentially erythrocyte-free plasma, at least one means to isolate the thrombocyte-containing plasma, at least one means to separate the thrombocyte-containing plasma into a thrombocyte fraction and into a supernatant of thrombocyte-poor plasma by means of centrifugation, at least one means to remove supernatant from thrombocyte-poor plasma, at least one means to mix the thrombocyte-poor plasma that was removed, at least one means to reapply mixed thrombocyte-poor plasma into the thrombocyte fraction, at least one means to resuspend the thrombocyte fraction in the reapplied thrombocyte-poor plasma and/or at least one means to obtain the plasma rich specifically in activated thrombocytes. In a preferred variant, the kit further contains at least one means to coagulate the thrombocyte-rich plasma into a thrombocyte-rich gel and means to obtain the thrombocyte-rich gel.

A further subject of the invention is a kit, specifically to obtain plasma rich specifically in activated thrombocytes from whole blood, containing the aforementioned device in accordance with the invention and a centrifuge, in particular with centrifuge inserts adapted to the device in accordance with the invention, specifically centrifuge buckets including balance chambers. Preferably the kit in accordance with the invention contains a centrifuge which has been modified to use the device in accordance with the invention. The modification consists specifically of a special rotor and specifically four special hangers having at least two metal buckets plus metal screw-down cover which can all be sterilized each time, and at least one non-sterile metal bucket including metal screw-down cover for the weight balance. To carry out the method in accordance with the invention, two sterile shrink wrapped with solid metal hangers consisting of metal bucket and metal screw-down cover are required. These metal hangers are designed so that they can be sterilized by means of steam sterilization.

The invention is explained in more detail with reference to the following drawings and examples. Additional embodiments will become clear from the dependent claims.

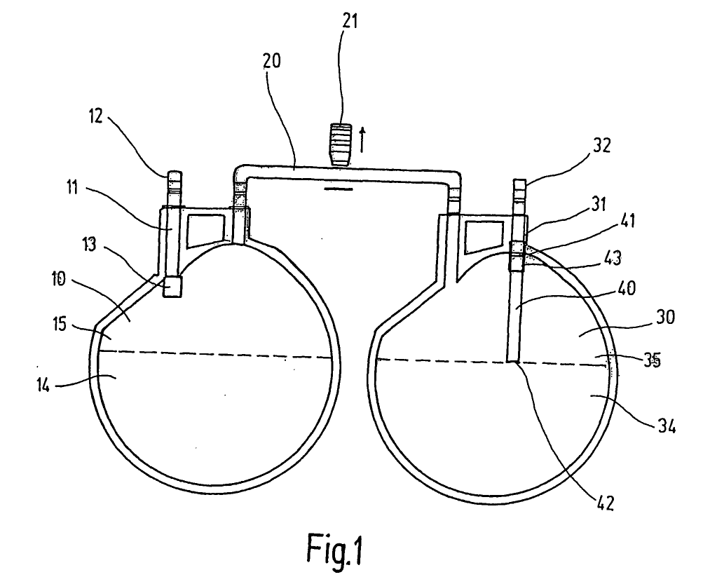

FIG. 1 shows a schematic representation of a preferred embodiment of the device in accordance with the invention, consisting of a primary chamber (10) configured as a flexible bag having a semicircular-shaped lower section (14) and a tapering funnel-shaped upper section (15) with at least one delivery and/or discharge (11) which issues into the funnel-shaped upper section (15) of the primary chamber and at its lower end, which issues into the lumen of the primary chamber (10), carries a lip valve (13), meaning a flutter valve, and at its upper end, outside the primary chamber, is provided with a connection (12) designed as a Luerlock. The primary chamber (10) further has a transfer line (20) which opens at the peak of the funnel-shaped upper section of the primary chamber (10). This transfer line (20) constitutes a closable connection between the volume of the primary chamber (10) and the volume of the secondary chamber (30). The flexible transparent transfer line (20) is closed by the interrupt (21) which is configured as a slide. The secondary chamber (30), designed as a flexible bag, consists of a semicircular-shaped lower section (34) and a tapering, funnel-shaped upper section (35) and a delivery and/or discharge (31) which issues at the peak of the funnel-shaped tapering upper section (35) of the secondary chamber (30) into the lumen of the secondary chamber and at its lower end, which is configured as a riser tube adapter (43), it has a riser tube (40) and at its upper end it has a connection (32) which is designed as a Luerlock. The riser tube (40) has an upper opening (41) and a lower opening (42). The upper opening (41) is located immediately at the peak of the funnel-shaped tapering upper section of the secondary chamber (30) at the riser tube adapter (43). The lower opening (42) is located at the lower end, approximately in the middle of the lumen of the secondary chamber (30) in the area of the transition between the semicircular-shaped lower section (34) and the funnel-shaped upper section (35).

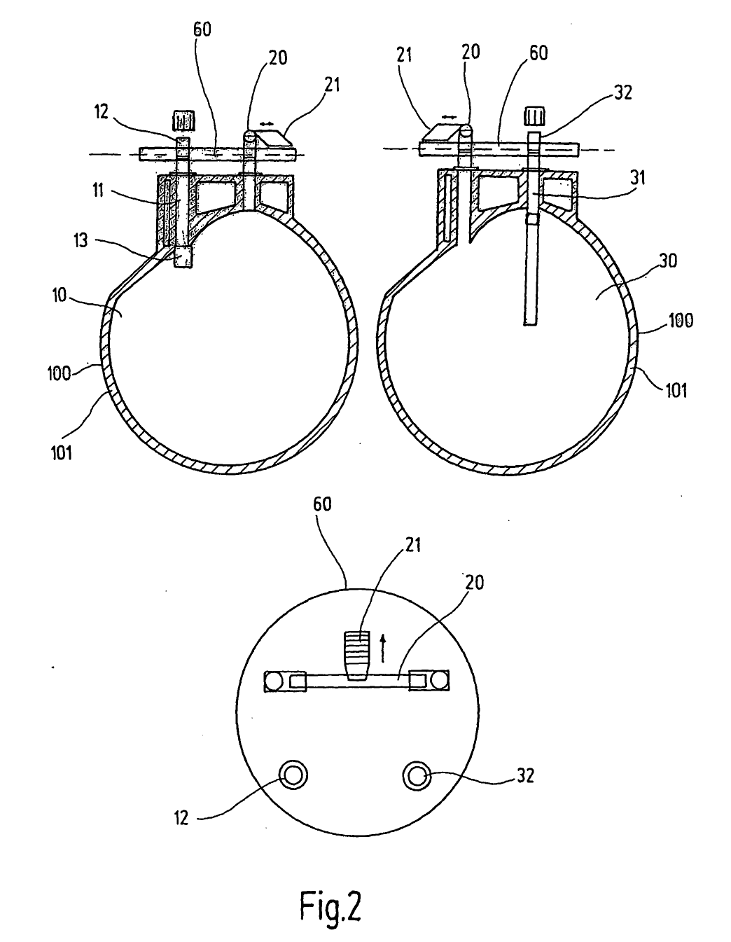

FIG. 2 shows a further preferred embodiment of the primary chamber (10), or the secondary chamber respectively (30), of the device in accordance with the invention which is designed as a flexible, flat bag. The bags are made from two plastic sheets laid over one another, which when laid over one another are cut out at the line 100 and welded over the surface 101. Primary chamber (10) and secondary chamber (30) and the delivery and/or discharge (11, 31) with the connections (12, 32) and the transfer line (20) are mounted on a carrier plate (60). The transfer line (20) is designed as a flexible hose and is closed by the interrupt (21) configured as a slide clamp which is moveably disposed on the carrier plate (60).

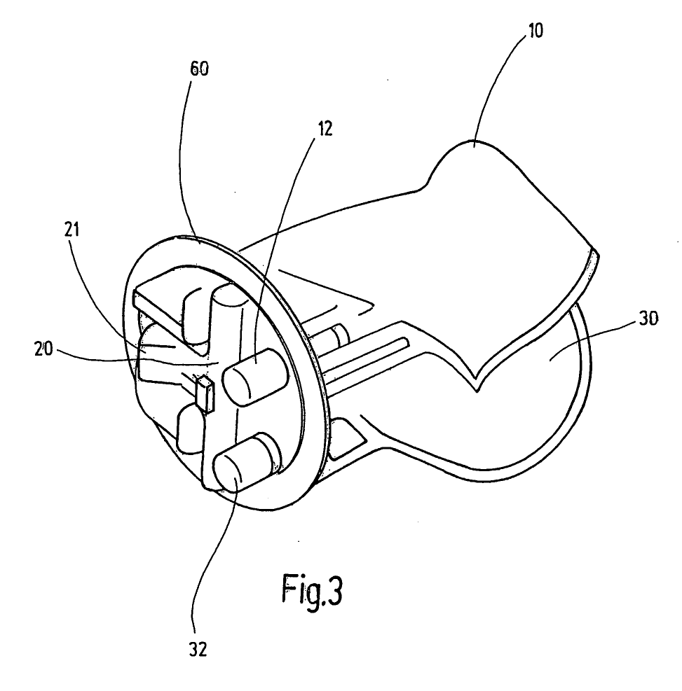

FIG. 3 shows a preferred embodiment of the device in accordance with the invention.

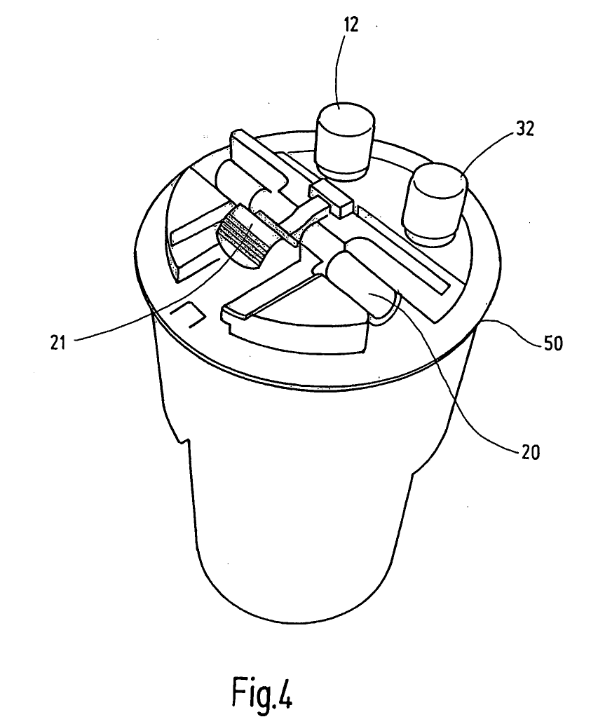

FIG. 4 shows the embodiment from FIG. 3, set into a metal centrifuge bucket (50).

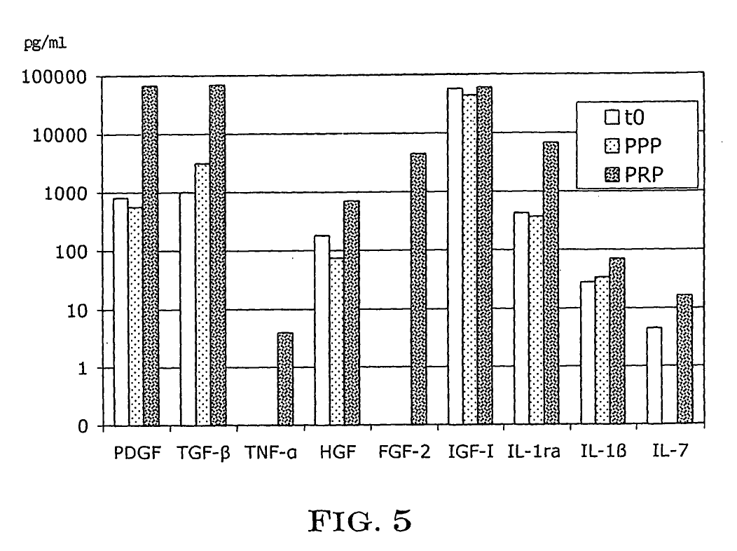

FIG. 5 shows the results (numbers on the ordinate in pg proteins/ml) from ELISA tests on various growth factors, or cytokines in serum obtained from whole blood immediately after it was drawn (Legend: t0), thrombocyte-poor plasma (Legend: PPP) separated in accordance with the invention from whole blood and in coagulated gel obtained in accordance with the invention, specifically from thrombocyte-rich gel(Legend: PRP).

### EXAMPLE 1

Kit for Obtaining Thrombocyte-Rich Plasma from Whole Blood

A sterilizable kit for disposable use is assembled which contains the following:

The bag system in accordance with the invention to produce thrombocyte concentrate (FIGS. 1 to 3, Table 1),

One 20-gauge cannula to draw up the ACD-A (anticoagulant) into the syringe for drawing the blood,

One 60-ml syringe for drawing blood,

One butterfly cannula for drawing blood,

One 60-ml syringe for receiving thrombocyte-poor plasma,

One 10-ml syringe for receiving thrombocyte-rich plasma,

One citrate/dextrose solution as anticoagulant at 6-ml per ampoule (ACD-A),

One 10-ml ampoule with 10% calcium gluconate,

One ampoule with 1000 I.U. bovine thrombin.

All the components are disposable articles, packaged and gamma-sterilized and provided as a whole with sterile outer packaging.

Tables 1 and 2 list the materials of the components used.

TABLE 1
Device in accordance with the invention from FIGS. 1 to 3

| Component: | Material, Supplier |
|---|---|
| Carrier plate (60) (cover) | ABS Terlux 2802 translucent |
| | green, |
| Primary chamber as bag (10) | Bag plastic: |
| | PVC compound 3222 from |
| | Solvay Draka |
| Secondary chamber as bag (30) | Bag plastic: |
| | PVC compound 3222 from |
| | Solvay Draka |
| Riser tube (40) | s 12/1 of PVC Raumedic 7567 |
| | Dimensions: 2.2 × 4.15 mm |
| Riser tube adapter (43) | ABS Terlux 2802, natural |
| Hose for transfer line (20) | PVC RB4 NDG 75 Shore A, |
| | 3.0 × 4.1 mm |
| Angle for transfer line (20) | PVC RB 1S2 |
| Interrupter (21) as slide valve | ABS Terlux 2802, white |
| Luerlock (LL), female, red for | ABS Terlux 2802, translucent |
| connection (32) | red, |
| Luerlock (LL), female for | ABS Terlux 2802, natural |
| connection (32) | |

TABLE 2
Kit

| Component: | Material, Supplier |
|---|---|
| Device in accordance with invention | (see Table 1 ) |
| Butterfly cannula 1.1 × 19 mm | Sealing cap: PE |
| | LL adapter: ABS transparent |
| | Wing attaching head: PVC |
| | Hose: PVC 60 Sh A |
| | Cannula: ISO 638/13 |
| | Protective hose: PE |
| Cannula 1.1 × 40 mm | Attaching head: PP |
| | Protective cap: PP |
| | Cannula: Stainless |
| | steel meeting DIN EN ISO |
| | 9626 |
| 60-ml syringe | Barrel: PP |
| | Plunger: PP |
| | Piston stopper: Natural |
| | rubber |
| 10-ml syringe (12 cc) | Barrel: PP |
| | Plunger: PP |
| | Piston stopper: Natural |
| | rubber |
| Perfusor line 1.5 m, 1.0 × 2.7 | LL male: ABS KR 2802 |
| mm | Cap: PE, opaque |
| | LL female: PVC |
| | Cap: ABS, red |
| | Hose: |
| | inner layer: ND PE |
| | middle layer: EVA |
| | outer layer: PVC |
| Blister pack | PET-GAG |
| 0.9 × 206 × 500 mm | |
| Tyvek sealing paper | Tyvek 10MP/1073B |

### EXAMPLE 2

Obtaining Thrombocyte-Rich Plasma from Whole Blood

a) Drawing Blood The required drawing of blood is performed using a 60-ml Luerlock syringe which is charged with 6 ml citrate/dextrose solution as an anticoagulant (ACD-A) before the blood is drawn. The syringe is filled slowly to the 60-mi mark with whole blood. Care is taken to ensure air- bubble free filling so that there is in fact exactly 60-ml in the syringe. Immediately after the blood is drawn, the blood filled syringe is swung back and forth 5 or 6 times to ensure that the ACD-A is evenly distributed.

b) Filling the Bag System and First Centrifugation

The interrupt (21) configured as a sliding clamp on the top side of the carrier surface is pushed to the center to close the hose. The sealing cap is removed and the syringe attached to the red connector (12). To prevent confusion between the two connectors on the carrier plate, the Luerlock connector for filling the whole blood is colored red. The contents of the syringe are filled slowly and completely through the delivery/discharge (11) into the primary chamber (10) which is configured as a flexible bag. The syringe is unscrewed after the filling process and the connector (12) of the bag is resealed with a new sealing cap.

The bag system with the filled primary chamber (10) is set into the empty sterile centrifuge hanger, into the centrifuge bucket (50). Care is taken to ensure that the carrier plate (60) of the bag system is correctly oriented in the bucket (FIG. 4). The centrifuge bucket (50) is closed with the appropriate screw-down cover. The sealed centrifuge bucket is set into the centrifuge. The bucket is held at a slight angle. After checking for the correct weight balance by means of a included water bottle (filled with about 30 ml water), centrifugation is carried out at 2500 rpm for 3 min. When centrifugation is complete, in which the cellular blood components are separated from the fluid components, the centrifuge bucket is carefully removed along with the bag system.

c) Separation of Erythrocytes and Plasma

As the result of centrifugation, the erythrocytes have collected in the lower section of the primary chamber (10). The supernatant plasma, as well as the buffy coat (mononuclear cells) and thrombocytes between them, while being checked visually are now transferred by slowly rolling up and/or expressing the primary chamber (10) by means of a conventional adjustable clamp from the bottom through the transfer line (20) into the secondary chamber (30), which is similarly designed as a flexible bag. The slide valve on the top side is pulled away again from the center of the cover beforehand so that the transparent hose of the transfer line (20) is released. As soon as the hose on the top side of the carrier plate is completely filled with red blood components and any few erythrocytes run into the secondary chamber, the slide valve interrupt (21) on the carrier plate is moved to the center in order to close the transfer hose and to stop the transfer to the secondary chamber.

d) Separation of Thrombocytes and Plasma

The secondary chamber now contains essentially the plasma component of the blood with thrombocytes and leukocytes as well as a small quantity of erythrocytes. The thrombocytes, however, are still evenly distributed in the plasma and are thus not sufficiently concentrated.

In a second centrifugation the thrombocytes (as well as the additional cellular blood components contained) are fractionated in the lower section of the bag as a pellet and the plasma fraction in the supernatant fractionated. To do this, the bag system is again placed in the centrifuge using a second sterile centrifuge bucket (50)and centrifuged at 3200 rpm for 15 min.

e) Obtaining Thrombocyte-Poor Plasma

At the end of the second centrifugation, the centrifuge bucket with the bag system is again removed from the centrifuge and the supernatant plasma (platelet-poor plasma PPP) is removed through the extraction connector (32) of the delivery/discharge (31) of the secondary chamber (30), except for a small remnant (less than about 2 ml). The syringe filled with plasma is unscrewed again.

f) Obtaining Thrombocyte-Rich Plasma

The thrombocyte-poor plasma that has been removed is thoroughly blended in the syringe. The syringe is emptied of air and attached to the extraction connector (32) again. About 4 ml of the plasma are returned to the bag. The plasma is blended with the thrombocyte fraction by lightly “massaging” the flexible bag so that a consistent thrombocyte suspension is formed. This is then removed from the bag using a 10-ml syringe through delivery/discharge (31). In order to ensure that the secondary chamber (30) is completely empty, it is turned upside down when the plasma is removed so that the remaining amount collects in the tip of the tapering section of the secondary chamber (30) and can be removed through the opening (41) of the collector tube (40) of the delivery/discharge (31) located there. The plasma thus obtained (quantity about 5 to 6 ml) is thrombocyte-rich plasma (PRP). g) Preparation of a Thrombocyte-Rich Gel

The thrombocyte-rich plasma (quantity about 5-6 ml) contained in the extraction syringe is brought to coagulation by the addition of 1 ml 10% calcium gluconate solution. The end product is about 6 to 7 ml of thrombocyte-rich gel which is distinguished by a high content of various growth factors (FIG. 5) and can be used for various applications. If only calcium ions are added, the clotting process takes about 10-15 min. If 1000 units of thrombin (bovine thrombin) are added in addition, clotting is clearly accelerated. Moreover, the effect of adding thrombin is a rapid cross linking of fibrin which causes the gel to adhere better to damaged tissue and thus results in improved applicability of the thrombocyte-rich gel.

h) Results

Thrombocyte Yield:

| | |
|---|---|
| Average number of thrombocytes in whole blood: | 275 × 10³/ml |
| Average number of thrombocytes in thrombocyte-poor | 19 × 10³/ml |
| plasma (PPP): | |
| Average number of thrombocytes in thrombocyte-rich | 1.3 × 10⁶/ml |
| plasma (PRP): | |

Yield of Cytokines and Growth Factors:

FIG. 5 shows the results of the ELISA tests.

It is clear from FIG. 5 that specifically the concentrations of growth factors PDGF, TGF-β, HGF, FGF-II and IL-1ra are substantially increased in thrombocyte-rich plasma (pg protein/ml) (logarithmic representation on the ordinate). The concentration of IGF-1 is clearly increased (logarithmic representation). Concentrations of TNF-α and IL-1β on the other hand scarcely increase at all.

1-10. (canceled)

11. A method for obtaining thrombocyte-rich plasma from whole blood comprising:
a) separating the whole blood into an erythrocyte-containing fraction and into essentially erythrocyte-free, thrombocyte-containing plasma;
b) isolating the thrombocyte-containing plasma;
c) separating the thrombocyte-containing plasma into a thrombocyte fraction and into a supernatant of thrombocyte-poor plasma by means of centrifugation;
d) removing supernatant from thrombocyte-poor plasma;
e) mixing the thrombocyte-poor plasma that was removed;
f) reapplying mixed thrombocyte-poor plasma to the thrombocyte fraction;
g) resuspending the thrombocyte fraction in the reapplied thrombocyte-poor plasma; and
h) obtaining the thrombocyte-rich plasma.

12. The method from claim 11, wherein separating the whole blood includes centrifugation and the erythrocyte-containing fraction is obtained as a pellet and the essentially erythrocyte-free and thrombocyte-containing plasma is obtained as a supernatant.

13. The method from claim 11, further including separating the thrombocyte-containing plasma from the erythrocyte-rich fraction by controlled transfer from a first chamber to a second chamber connected thereto, and stopping the transfer of the plasma when the incipient transfer of erythrocytes is detected.

14. The method from claim 11, further comprising coagulating the thrombocyte-rich plasma obtained a clotting agent and obtaining a gel rich in at least one of thrombocytes and growth.

15. A thrombocyte-rich plasma obtained in accordance the method from claim 11.

16. A thrombocyte-rich gel obtained in accordance with the method from claim 11.

17. A device for obtaining at least one of thrombocyte-rich plasma and gel from whole blood, the device comprising at least one primary chamber and at least one secondary chamber, each chamber having at least one of a closable discharge and a closable delivery which communicate over at least one sealable transfer line, the primary chamber and the secondary chamber configured in a pear shape from an essentially semicircular-shaped lower part and a funnel-shaped upper part narrowing toward the top.

18. The device from claim 17, wherein the at least one of a discharge and delivery of the secondary chamber has at least one riser tube extending into the secondary chamber with at least one lower opening formed at the boundary between semicircular-shaped lower part and funnel-shaped upper part of the chamber, and at least one upper opening preferably formed at an upper peak of the funnel-shaped upper part of the chamber.

19. A kit for obtaining thrombocyte-rich plasma from whole blood in accordance with a method from claim 11 and further comprising at least one means selected from the group including:
a) means to separate whole blood into an erythrocyte-containing fraction and a thrombocyte-containing, essentially erythrocyte-free plasma,
b) means to isolate the thrombocyte-containing plasma,
c) means to separate the thrombocyte-containing plasma into a thrombocyte fraction and into a supernatant from thrombocyte-poor plasma by means of centrifugation,
d) means to remove supernatant from thrombocyte-poor plasma,
e) means to mix the thrombocyte-poor plasma removed,
f) means to reapply mixed thrombocyte-poor plasma into the thrombocyte fraction,
g) means to resuspend the thrombocyte fraction in the reapplied thrombocyte-poor plasma and
h) means to obtain the thrombocyte-rich plasma.

20. The kit from claim 19 for obtaining thrombocyte-rich gel from whole blood, further comprising:
i) means to coagulate thrombocyte-rich plasma; and
j) means to obtain the thrombocyte-rich gel.

21. The method from claim 12, further comprising coagulating the thrombocyte-rich plasma obtained by a clotting agent, and obtaining a gel rich in at least one of thrombocytes and growth factors.

22. The method from claim 13, further comprising coagulating the thrombocyte-rich plasma obtained by a clotting agent, and obtaining a gel rich in at least one of thrombocytes and growth factors.

23. The thrombocyte-rich plasma obtained in accordance with the method from claim 12.

24. The thrombocyte-rich plasma obtained in accordance with the method from claim 13.

25. The thrombocyte-rich plasma obtained in accordance with the method from claim 14.

26. The thrombocyte-rich gel obtained in accordance with the method from claim 12.

27. The thrombocyte-rich gel obtained in accordance with the method from claim 13.

28. The thrombocyte-rich gel obtained in accordance with the method from claim 14.

29. The method from claim 21, wherein coagulating includes coagulating with a calcium gluconate solution.

30. The method from claim 22, wherein coagulating includes coagulating with a calcium gluconate solution.
